(19) United States
(12) Patent Application Publication
(10) Pub. No.: US 2023/0398166 A1
(43) Pub. Date: Dec. 14, 2023

(54) METHOD FOR EXTRACTING A FULL SPECTRUM OF HYDROPHILIC COMPOUNDS FROM BIOMASS

(71) Applicant: Jerred Tudela, Lebanon, OR (US)

(72) Inventor: Jerred Tudela, Lebanon, OR (US)

(21) Appl. No.: 18/207,903

(22) Filed: Jun. 9, 2023

Related U.S. Application Data
(60) Provisional application No. 63/350,804, filed on Jun. 9, 2022.

(51) Int. Cl. A61K 36/33; A61K 36/07

(52) U.S. Cl. CPC A61K 36/33; A61K 36/07; A61K 2236/15; A61K 2236/17; A61K 2236/333; A61K 2236/37; A61K 2236/53; A61K 2236/51

(57) ABSTRACT

This invention is directed toward a method for extracting a full spectrum of hydrophilic compounds from psychoactive biomass. A water-based solvent is used to extract the full spectrum hydrophilic compounds. The method involves contacting the psychoactive biomass with the water-based solvent to form a slurry. The slurry is allowed to steep; then, it is filtered and dried to provide a concentrate of the extracted hydrophilic compounds.

### CROSS-REFERENCE TO RELATED APPLICATIONS

This application claims priority to provisional application No. 63/350,804, filed Jun. 9, 2022.

### FEDERALLY SPONSORED RESEARCH AND DEVELOPMENT

Not applicable.

### PARTIES TO JOINT RESEARCH AGREEMENT

Not applicable.

### PRIOR DISCLOSURES BY INVENTOR OR JOINT INVENTOR

Not applicable.

### BACKGROUND

The field of this invention is related to the extraction of compounds from psychoactive biomass. The invention is directed to a method for extracting naturally occurring hydrophilic compounds from psychoactive biomass. This method uses water-based solvents to extract the hydrophilic compounds. The resulting extract contains the full spectrum of hydrophilic compounds from the psychoactive biomass. The full spectrum hydrophilic compounds include but are not limited to flavonoids, beta-carbolines, primary metabolites, secondary metabolites, antioxidants, plant colors, alkaloids, proto-alkaloids, lysergic amides, monoamine oxidase inhibitors, or any selection or combination thereof. The above naturally occurring full spectrum of hydrophilic compounds creates an entourage effect that does not lead to any side effects or adverse effects when ingested as a part of the composition.

The invention differs from the prior art in that it is water-based. On the other hand, the prior art teaches using lower aliphatic alcohol or a mixture of water and a water-soluble hydrocarbon solvent. Furthermore, the invention aims to extract the full spectrum of hydrophilic compounds. The prior art teaches further extraction using hydrocarbon solvents such as petroleum ether, acetone, or chloroform-alcohol. After such treatment, the residue is no longer a mixture of full spectrum of hydrophilic compounds.

The full spectrum of hydrophilic compounds from psychoactive biomass is a known practice for indigenous and shamanistic practitioners. Furthermore, the compounds have been found to have therapeutic utility. For example, preclinical and observational studies of ayahuasca users suggest that ayahuasca has anxiolytic, anti-depressive, and antiaddictive effects. Moreover, under the right-to-try laws, it is useful to provide a full spectrum of hydrophilic compounds for this clinical practice. Thus, it's beneficial to the art to have a method to extract a full spectrum of hydrophilic compounds without using aliphatic alcohol or hydrocarbon solvents.

### BRIEF SUMMARY

The method extracts the naturally occurring hydrophilic compounds by contacting the psychoactive biomass with a water-based solvent to make a slurry. The slurry is allowed to stand for a period, and then the slurry is filtered. The liquid filtrate collected. The filtrate is then dried, resulting in a concentrated version of the full spectrum of the hydrophilic compound.

Hydrophilic compounds are present in the full spectrum hydrophilic compound extract, ranging from 0.1-99.9% by dry weight. The concentration range of the naturally occurring hydrophilic compounds in the full spectrum hydrophilic compound extract will vary due to various factors, for example, but not limited to, the source of the full spectrum hydrophilic compound extract, the extraction technique used, the efficiency of the extraction process, and the amount of the hydrophilic compounds in the extract.

FIGS. 1-5 are process charts showing the basic steps of the extraction process. The flowchart indicates that a water-based solvent is added to psychoactive biomass. The psychoactive biomass may comprise but is not limited to dried, lyophilized or fresh, ground, or sliced raw fungal, plant, lichen, animal, bacterial, or cactus.

The water-based solvent may be water, citrus juice, or mixtures thereof. The amount of water-based solvent used varies according to the weight of the psychoactive biomass. A wide range of solvent-to-psychoactive psychoactive biomass ratios can be used. Typically, a 1:1 to 50:1 water-based solvent-psychoactive psychoactive biomass ratio may be used for the extraction.

As a result of adding the water-based solvent and soaking the psychoactive biomass of dried, lyophilized, or fresh raw psychoactive biomass in the water-based solvent, the hydrophilic compounds in the psychoactive biomass dissolve into the water-based solvent. The water-based solvent may be at a low or high temperature, and pressure may be applied. The optimal temperature and pressure depend on the hardiness of the psychoactive biomass and on the type of solvent used for the process. However, the preferred temperature for extraction is 4-85° C. The useful temperature range spans most of the liquid state of the solvent used. Still, the temperature of the water-based solvent may be outside of this range in other embodiments. In some embodiments, when pressure is applied, the temperature can reach 107° C. The duration of the extraction is from 10 minutes to 24 hours, with or without agitation. The optimum time is determined by experimentation and depends on the water-based solvent and degree of agitation. FIGS. 3-5 provide the preferred embodiments for extraction from low-density, medium-density, and animal-based psychoactive biomass.

### BRIEF DESCRIPTION OF THE DRAWINGS

FIG. 1 is an overview of the extraction process.

FIG. 2 is a general description of the extraction process.

FIG. 3 describes the extraction process for low-density psychoactive biomass.

FIG. 4 describes the extraction process for medium-density psychoactive biomass.

FIG. 5 describes the extraction process for animal-based psychoactive biomass.

FIG. 6 is a photograph of a DMT containing filtrate from three extractions.

FIG. 7 is a photograph of the dried DMT extract.

### DETAILED DESCRIPTION

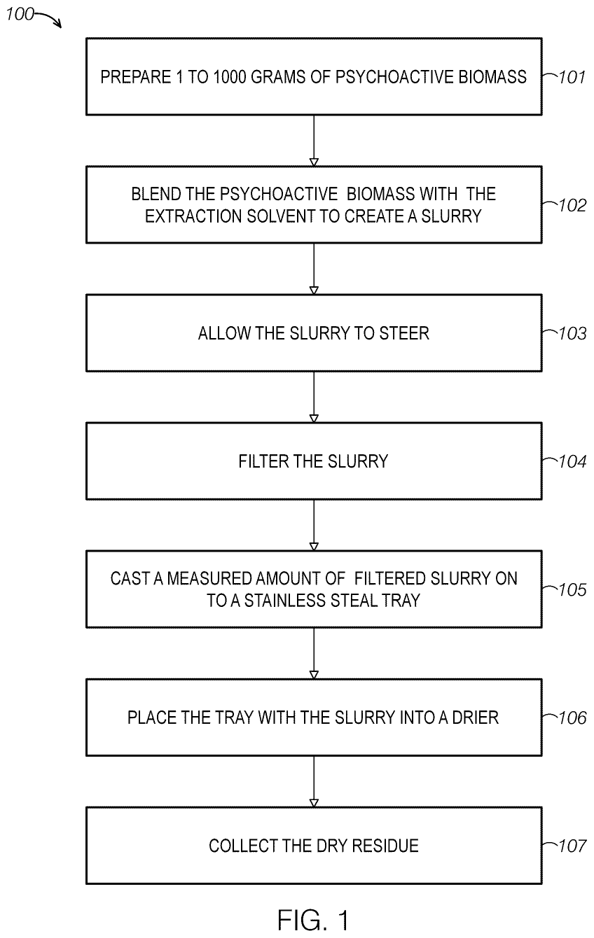

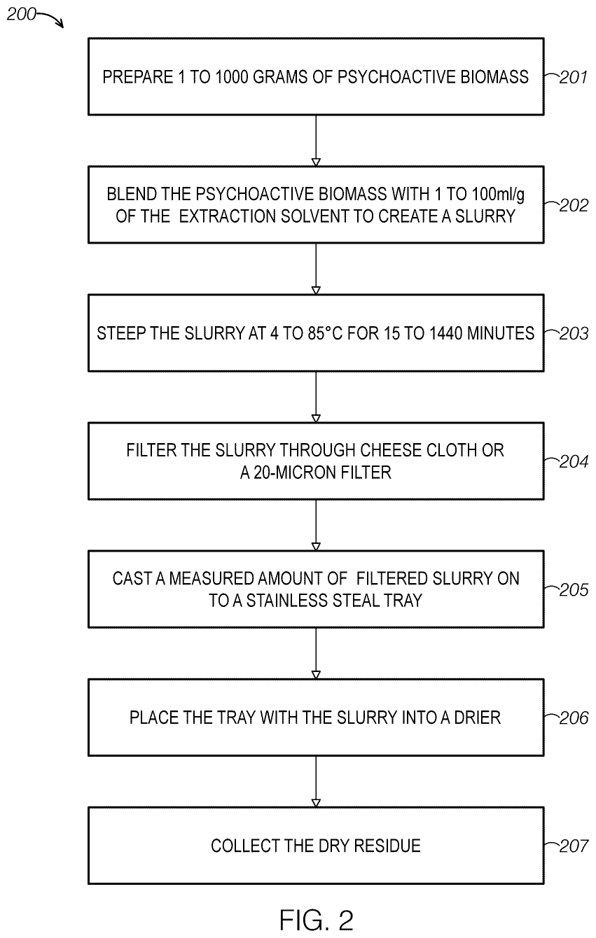

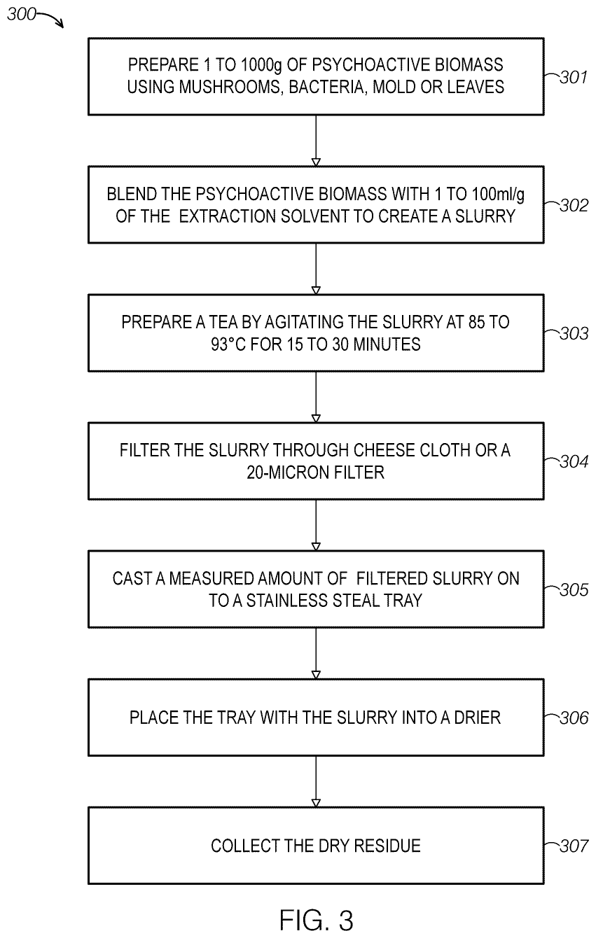

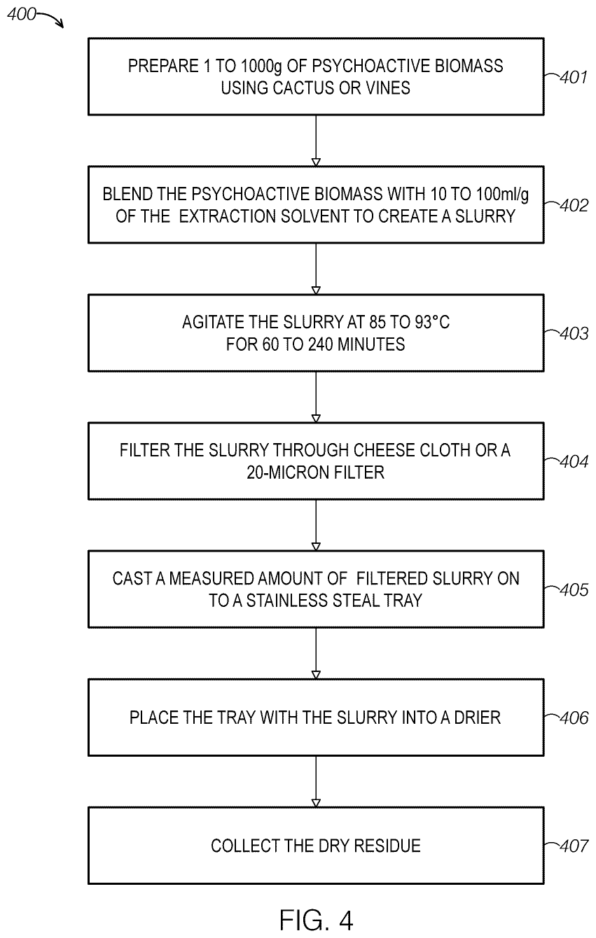

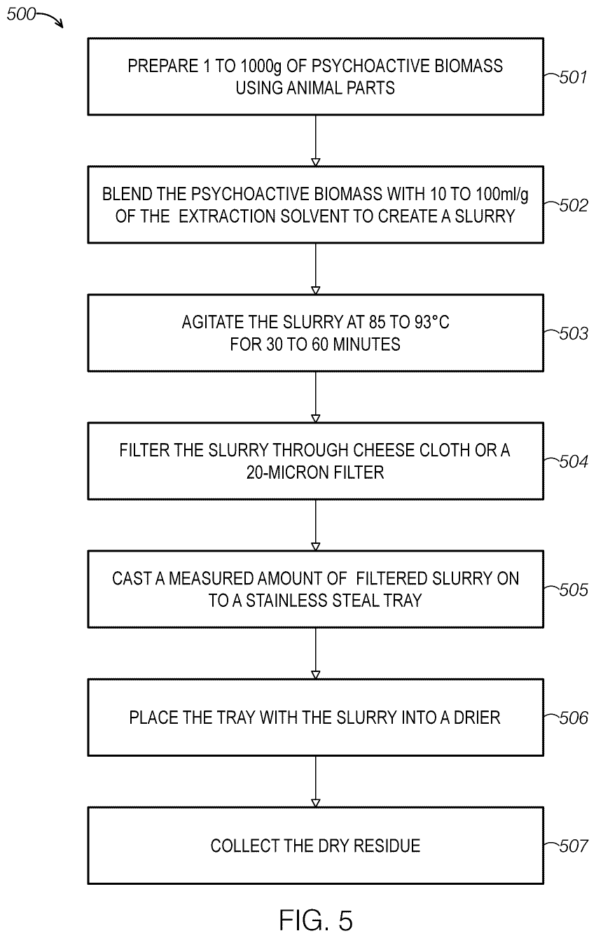

FIGS. 1 and 2 provide an overview of the extraction process. The method for extracting full spectrum hydrophilic compounds comprises: (I) providing a quantity of psychoactive biomass; (II) drying the psychoactive biomass; (III) reducing the size of the psychoactive biomass; (IV) making a slurry by contacting the psychoactive biomass with a solvent; extracting the hydrophilic compounds by steeping the slurry for a period of time; (VI) filtering the slurry and collecting the filtrate; (VII) drying the filtrate; and (VIII) collecting the extract. The process may be repeated using the same sample of biomass. Optionally the slurry's temperature can be maintained between 4 to 85° C.; pressure may be applied to the slurry; the slurry can be pressurized to 70 to 140 kPa.

The percentage of hydrophilic compounds may be standardized by combining psychoactive biomass with a high concentration of hydrophilic compounds with psychoactive biomass having a lower concentration of hydrophilic compounds. The blend is a ratio that yields a standardized amount of hydrophilic compound. Another method for standardization is to blend extracts from successive runs of the process. The first extraction will provide a high-concentration extract, and the following extracts will be lower in concentration. The extracts are then blended in a ratio that provides a standardized amount.

### Step I: Psychoactive Biomass

Psychoactive biomass is the base material from which the hydrophilic compounds are extracted. Sources of psychoactive biomass include fungi, plants, lichens, animals, bacteria, and mold. The psychoactive biomass may come in many forms, such as dried, lyophilized, or fresh. The whole plant, or sometimes only the leaves, vines, bark, seeds, or roots, are used. Any part of the mushroom may be used for fungi, e.g., caps, gills, stems, and mycelium. For cacti, whole columns or only the rind or pulp can be used. For animals, the entire animal or only the venom or other individual parts are used. Additionally, parts of the raw fungal, plant, lichen, animal, bacterial, or cactus that are typically considered waste have valuable active hydrophilic compounds and may be used.

For fungi, mushrooms that contain psilocybin and psilocin are preferred. Non-limiting examples include species of the following genera: Psilocybin, Panaeolus, Gymnopilus, Conocybe, Galerina, Inocybe, Pholiotina, and Plutes. Useful non-limiting examples of psilocybin and psilocin-containing mushrooms include: Psilocybe azurescens, Psilocybe bohemica, Psilocybe baeocystis, Psilocybe cubenis, Psilocybe cyanescens, Psilocybe hoogshagenii, Psilocybe semilanceata, Psilocybe stuntzii, Psilocybe tampanensis, Psilocybe liniformans, and Psilocybe weilii. In addition to psilocybin and psilocin, fungi that contain muscimol are also preferred. Non-limiting examples are species of the genus Amanita are, Amanita muscaria and Amanita pantherine being preferred. Lastly, fungi of the Ergot genus, most notably Claviceps purpurea, provide alkaloids that have psychoactive properties are preferred.

Plants include species that contain tryptamines such as N,N-Dimethyltryptamine (DMT) are useful. Non-limiting examples of DMT-containing species are Psychotria viridis, Diplopterys cabrerana, Psychotria carthagenensis, and Mimosa tenuiflora. Additionally, it's preferable to blend plants having DMT with plants containing monoamine oxidase inhibitors (MAOI). Non-limiting examples of MAOI-containing species include Peganum harmala (Syrian Rue), Banisteriopsis caapi, Passiflora incarnata, Kaempferia galangal, and Tribulus terrestris. Typically blends of plants with MAOI and DMT are made to make ayahuasca. Ayahuasca is a drink used in shamanistic practices of the indigenous peoples of South America.

Cacti provides useful biomass—mescaline-containing cacti are preferred. Non-limiting examples include Peyotes such as Lophophora williamsii and Lophophora decipiens; Echinopsis such as Echinopsis pachanoi (San Pedro), Echinopsis zamnesiana, and Echinopsis lageniformis (Bolivian torch cactus).

Plants such as Rivea corymbosa, Ipomoea violacea (Morning Glory), and Argyreia nervosa (Hawaiian Baby Woodrose) species provide seeds containing d-lysergic acid amide, or Ergine (LSA). Other plants include Kava root, which provides kavapyrones (kavalactones), Kratom, which provides mitragynine and 7-hydroxy mitragynine; and Erythroxylum coca.

Lastly, psychedelic fauna can provide psychoactive biomass. One example is fish. Non-limiting examples include Sarpa salpa, Kyphosus fuscus, Kyphosus cinerascens, and Kypphosus viagiensis.

### Step II: Drying

Before extraction, the psychoactive biomass may be dried. Air drying, heat drying, or lyophilization methods may be used. Lyophilized or fresh psychoactive biomass is preferred because it lends itself well to extraction and preserves the hydrophilic compounds without degradation. The temperature of the oven or lyophilization device and the drying time depend on how much moisture is in the raw psychoactive biomass and the quantity of raw psychoactive biomass. The moisture content of the raw psychoactive biomass should be between 0%-95%. The moisture for air or heat-dried psychoactive biomass should be less than 5%, and 0% for lyophilized psychoactive biomass. On the other hand, wet psychoactive biomass, such as cactus, with moisture above 80%, is preferred.

### Step III: Size Reduction

After drying, the psychoactive biomass may be reduced in size to granules or powders by grinding, milling, or pulverizing. The dried psychoactive biomass is reduced to 6 mm to 0.03 mm particle size in one embodiment.

In an another embodiment, the dried psychoactive biomass is reduced to a particle size of about 6 mm to about 5 mm, about 6 mm to about 4 mm, about 6 mm to about 3 mm, about 6 mm to about 2 mm, about 6 mm to about 1 mm, about 6 mm to about 0.5 mm, about 6 mm to about 0.1 mm, about 6 mm to about 0.05 mm, about 6 mm to about 0.03 mm, about 5 mm to about 4 mm, about 5 mm to about 3 mm, about 5 mm to about 2 mm, about 5 mm to about 1 mm, about 5 mm to about 0.5 mm, about 5 mm to about 0.1 mm, about 5 mm to about 0.05 mm, about 5 mm to about 0.03 mm, about 4 mm to about 3 mm, about 4 mm to about 2 mm, about 4 mm to about 1 mm, about 4 mm to about 0.5 mm, about 4 mm to about 0.1 mm, about 4 mm to about 0.05 mm, about 4 mm to about 0.03 mm, about 3 mm to about 2 mm, about 3 mm to about 1 mm, about 3 mm to about 0.5 mm, about 3 mm to about 0.1 mm, about 3 mm to about 0.05 mm, about 3 mm to about 0.03 mm, about 2 mm to about 1 mm, about 2 mm to about 0.5 mm, about 2 mm to about 0.1 mm, about 2 mm to about 0.05 mm, about 2 mm to about 0.03 mm, about 1 mm to about 0.5 mm, about 1 mm to about 0.1 mm, about 1 mm to about 0.05 mm, about 1 mm to about 0.03 mm, about 0.5 mm to about 0.1 mm, about 0.5 mm to about 0.05 mm, about 0.5 mm to about 0.03 mm, about 0.1 mm to about 0.05 mm, about 0.1 mm to about 0.03 mm, or about 0.05 mm to about 0.03 mm.

In another embodiment, the psychoactive biomass was reduced to a particle size of about 6 mm, about 5 mm, about 4 mm, about 3 mm, about 2 mm, about 1 mm, about 0.5 mm, about 0.1 mm, about 0.05, or about 0.03 mm.

In other embodiments, the psychoactive biomass is reduced to a particle size of at least 6 mm, about 5 mm, about 4 mm, about 3 mm, about 2 mm, about 1 mm, about 0.5 mm, about 0.1 mm, or about 0.05 mm.

In some embodiments, the psychoactive biomass is reduced to a particle size of about 5 mm, about 4 mm, about 3 mm, about 2 mm, about 1 mm, about 0.5 mm, about 0.1 mm, about 0.05 mm, or about 0.03 mm.

In some embodiments, the psychoactive biomass is reduced to a particle size of 1 mm to 0.03 mm before contact with the waterbased solvent.

In some embodiments, the psychoactive biomass is reduced to a particle size of about 0.03 mm to about 1 mm.

In some embodiments the psychoactive biomass is reduced to a particle size of about 1 mm to about 0.9 mm, about 1 mm to about 0.8 mm, about 1 mm to about 0.7 mm, about 1 mm to about 0.6 mm, about 1 mm to about 0.5 mm, about 1 mm to about 0.4 mm, about 1 mm to about 0.3 mm, about 1 mm to about 0.2 mm, about 1 mm to about 0.1 mm, about 1 mm to about 0.05 mm, about 1 mm to about 0.03 mm, about 0.9 mm to about 0.8 mm, about 0.9 mm to about 0.7 mm, about 0.9 mm to about 0.6 mm, about 0.9 mm to about 0.5 mm, about 0.9 mm to about 0.4 mm, about 0.9 mm to about 0.3 mm, about 0.9 mm to about 0.2 mm, about 0.9 mm to about 0.1 mm, about 0.9 mm to about 0.05 mm, about 0.9 mm to about 0.03 mm, about 0.8 mm to about 0.7 mm, about 0.8 mm to about 0.6 mm, about 0.8 mm to about 0.5 mm, about 0.8 mm to about 0.4 mm, about 0.8 mm to about 0.3 mm, about 0.8 mm to about 0.2 mm, about 0.8 mm to about 0.1 mm, about 0.8 mm to about 0.05 mm, about 0.8 mm to about 0.03 mm, about 0.7 mm to about 0.6 mm, about 0.7 mm to about 0.5 mm, about 0.7 mm to about 0.4 mm, about 0.7 mm to about 0.3 mm, about 0.7 mm to about 0.2 mm, about 0.7 mm to about 0.1 mm, about 0.7 mm to about 0.05 mm, about 0.7 mm to about 0.03 mm, about 0.6 mm to about 0.5 mm, about 0.6 mm to about 0.4 mm, about 0.6 mm to about 0.3 mm, about 0.6 mm to about 0.2 mm, about 0.6 mm to about 0.1 mm, about 0.6 mm to about 0.05 mm, about 0.6 mm to about 0.03 mm, about 0.5 mm to about 0.4 mm, about 0.5 mm to about 0.3 mm, about 0.5 mm to about 0.2 mm, about 0.5 mm to about 0.1 mm, about 0.5 mm to about 0.05 mm, about 0.5 mm to about 0.03 mm, about 0.4 mm to about 0.3 mm, about 0.4 mm to about 0.2 mm, about 0.4 mm to about 0.1 mm, about 0.4 mm to about 0.05 mm, about 0.4 mm to about 0.03 mm, about 0.3 mm to about 0.2 mm, about 0.3 mm to about 0.1 mm, about 0.3 mm to about 0.05 mm, about 0.3 mm to about 0.03 mm, about 0.2 mm to about 0.1 mm, about 0.2 mm to about 0.05 mm, about 0.2 mm to about 0.03 mm, about 0.1 mm to about 0.05 mm, about 0.1 mm to about 0.03 mm, or about 0.05 mm to about 0.03 mm.

In some embodiments, the psychoactive biomass is reduced to a particle size of about 1 mm, about 0.9 mm, about 0.8 mm, about 0.7 mm, about 0.6 mm, about 0.5 mm, about 0.4 mm, about 0.3 mm, about 0.2 mm, about 0.1 mm, about 0.05 mm, or about 0.03 mm. In some embodiments, before contact with the waterbased solvent, the psychoactive biomass of the fungal, plant, lichen, animal, bacterial, or cactus organism is reduced to a particle size of at least about 1 mm, about 0.9 mm, about 0.8 mm, about 0.7 mm, about 0.6 mm, about 0.5 mm, about 0.4 mm, about 0.3 mm, about 0.2 mm, about 0.1 mm, or about 0.05 mm.

In some embodiments, the psychoactive biomass is reduced to a particle size of at most about 0.9 mm, about 0.8 mm, about 0.7 mm, about 0.6 mm, about 0.5 mm, about 0.4 mm, about 0.3 mm, about 0.2 mm, about 0.1 mm, about 0.05 mm, or about 0.03 mm.

For fresh or high-moisture psychoactive biomass, the material size may be reduced by slicing, dicing, or shredding. In one embodiment, the psychoactive biomass is sliced to at least 0.25 cm in width.

### Step IV: Preparing the Slurry

The hydrophilic compounds are extracted by contacting the prepared psychoactive biomass with an extraction solvent forming a slurry. The extraction solvent is a water-based solvent comprising water, citrus juice, and mixtures thereof. The solvents used to extract hydrophilic compounds are water, lemon juice, lime juice, grapefruit juice, and any combination thereof. In some embodiments, only water is used. In some embodiments, the water is selected from deionized, distilled, reverse osmosis, or otherwise purified water, substantially without free ions. In other embodiments, the water is not purified.

A slurry is made by mixing the water-based solvent with the psychoactive biomass, using a solvent-psychoactive biomass ratio of 1:1 to 50:1. The amount of solvent used depends on the weight of the psychoactive biomass. In some embodiments, the extraction is performed with an water-based solvent-to-solid ratio of 10 to 100 ml/g, wherein the solid is dried, lyophilized, or fresh psychoactive biomass. In one embodiment, the extraction is performed with an water-based solvent to a solid ratio of 20 ml/g. In some embodiments, the extraction is performed with an water-based solvent to a solid ratio of about 10 ml/g to about 100 ml/g.

In some embodiments, the extraction is performed with an water-based solvent to solid ratio in the range of about 10 ml/g to about 20 ml/g, about 10 ml/g to about 30 ml/g, about 10 ml/g to about 40 ml/g, about 10 ml/g to about 50 ml/g, about 10 ml/g to about 60 ml/g, about 10 ml/g to about 70 ml/g, about 10 ml/g to about 80 ml/g, about 10 ml/g to about 90 ml/g, about 10 ml/g to about 100 ml/g, about 20 ml/g to about 30 ml/g, about 20 ml/g to about 40 ml/g, about 20 ml/g to about 50 ml/g, about 20 ml/g to about 60 ml/g, about 20 ml/g to about 70 ml/g, about 20 ml/g to about 80 ml/g, about 20 ml/g to about 90 ml/g, about 20 ml/g to about 100 ml/g, about 30 ml/g to about 40 ml/g, about 30 ml/g to about 50 ml/g, about 30 ml/g to about 60 ml/g, about 30 ml/g to about 70 ml/g, about 30 ml/g to about 80 ml/g, about 30 ml/g to about 90 ml/g, about 30 ml/g to about 100 ml/g, about 40 ml/g to about 50 ml/g, about 40 ml/g to about 60 ml/g, about 40 ml/g to about 70 ml/g, about 40 ml/g to about 80 ml/g, about 40 ml/g to about 90 ml/g, about 40 ml/g to about 100 ml/g, about 50 ml/g to about 60 ml/g, about 50 ml/g to about 70 ml/g, about 50 ml/g to about 80 ml/g, about 50 ml/g to about 90 ml/g, about 50 ml/g to about 100 ml/g, about 60 ml/g to about 70 ml/g, about 60 ml/g to about 80 ml/g, about 60 ml/g to about 90 ml/g, about 60 ml/g to about 100 ml/g, about 70 ml/g to about 80 ml/g, about 70 ml/g to about 90 ml/g, about 70 ml/g to about 100 ml/g, about 80 ml/g to about 90 ml/g, about 80 ml/g to about 100 ml/g, or about 90 ml/g to about 100 ml/g.

In some embodiments, the extraction is performed with an water-based solvent to solid ratio of about 10 ml/g, about 20 ml/g, about 30 ml/g, about 40 ml/g, about 50 ml/g, about 60 ml/g, about 70 ml/g, about 80 ml/g, about 90 ml/g, or about 100 ml/g. In some embodiments, extraction is performed with an water-based solvent to solid ratio of at least about 10 ml/g, about 20 ml/g, about 30 ml/g, about 40 ml/g, about 50 ml/g, about 60 ml/g, about 70 ml/g, about 80 ml/g, or about 90 ml/g. In some embodiments, the extraction is performed with an water-based solvent-to-solid ratio in the range of at most about 20 ml/g, about 30 ml/g, about 40 ml/g, about 50 ml/g, about 60 ml/g, about 70 ml/g, about 80 ml/g, about 90 ml/g, or about 100 ml/g.

The resulting slurry contains undissolved psychoactive biomass solids, the water-based solvent, and dissolved full-spectrum hydrophilic compounds. Some of the undissolved solids may be undesirable components and can be discarded. The undissolved solids may be used for several more extractions. In some embodiments, extractions from previously extracted psychoactive biomass low in active hydrophilic compounds and high in inactive hydrophilic compounds may be used as placebo extracts.

### Step V: Steeping

The hydrophilic compounds are extracted by steeping the slurry. The time varies; in some embodiments, the slurry can be steeped for a duration of 10-24 hours, depending on the hardiness of the psychoactive biomass, material to be extracted, and the temperature of the slurry. For most cases, less than 10 minutes is insufficient, and longer than 24 hours may damage sensitive compounds. The slurry may be agitated, simmered, boiled, or left quiet.

In some embodiments, the extraction is performed for a period ranging from about 10 minutes to about 24 hours. In some embodiments, the extraction is performed for a period ranging from about 10 min to about 100 min, about 10 min to about 200 min, about 10 min to about 300 min, about 10 min to about 400 min, about 10 min to about 500 min, about 10 min to about 600 min, about 10 min to about 720 min, about 100 min to about 200 min, about 100 min to about 300 min, about 100 min to about 400 min, about 100 min to about 500 min, about 100 min to about 600 min, about 100 min to about 720 min, about 200 min to about 300 min, about 200 min to about 400 min, about 200 min to about 500 min, about 200 min to about 600 min, about 200 min to about 720 min, about 300 min to about 400 min, about 300 min to about 500 min, about 300 min to about 600 min, about 300 min to about 720 min, about 400 min to about 500 min, about 400 min to about 600 min, about 400 min to about 720 min, about 500 min to about 600 min, about 500 min to about 720 min, or about 600 min to about 720 min.

In some embodiments, the extraction is performed for a period ranging from about 10 min about 100 min, about 200 min, about 300 min, about 400 min, about 500 min, about 600 min, or about 720 min. In some embodiments, the extraction is performed for a period ranging from at least about 10 min, about 100 min, about 200 min, about 300 min, about 400 min, about 500 min, or about 600 min. In some embodiments, the extraction is performed for a period ranging from about 100 min about 200 min, about 300 min, about 400 min, about 500 min, about 600 min, or about 720 min. In some embodiments, extraction is performed for 24 hours. In another embodiment, and more usually, the extraction is performed for a period ranging from 30 min to about 2 hours.

### Step VI: Filtration

After extraction, the slurry is filtered, and the filtrate is collected. The filter can be coarse, like cheesecloth, or fine, like a 20-micron filter. After filtration, the result is a liquid extract free of large particles. The filtering step may be carried out when the extraction slurry is cold, or it may be carried out while still hot. The filtration steps may be repeated on the same slurry. The hydrophilic compounds may be concentrated by evaporating some of or all of the water-based solvent from the filtrate. The resulting filtrate contains a full spectrum of dissolved active and inactive hydrophilic compounds.

### Step VII: Drying

The filtrate can be dried by air, heat, or freeze-drying. If stored correctly, freeze-drying the filtrate removes the waterbased solvent while preserving the purified hydrophilic compounds in a non-perishable form.

### Step VIII: The Extract

The extract is a dry powdered, full-spectrum, hydrophilic extract that has a total active compound concentration of 0.1-10% by dry weight. Other inactive compounds which help build a full spectrum may be present in the extract. These may be flavonoids, sugars, proteins, carbohydrates, fats, and other nutraceutical compounds, comprising about half of the extract. Drying the filtrate is optional as it may be intended to produce a liquid extract instead of a dry, powdered extract.

### Option A: Temperature

The extraction process described above can be run at elevated temperatures. In some embodiments, the extraction is performed at a temperature ranging from about 4° C. to about 10° C., about 4° C. to about 16° C., about 4° C. to about 21° C., about 4° C. to about 27° C., about 4° C. to about 32° C., about 4° C. to about 38° C., about 4° C. to about 43° C., about 4° C. to about 49° C., about 4° C. to about 54° C., about 4° C. to about 60° C., about 4° C. to about 66° C., about 4° C. to about 71° C., about 4° C. to about 76° C., about 4° C. to about 82 ° C., about 4° C. to about 85° C. In some embodiments, the extraction is performed at 4-85° C.

In other embodiments, the extraction is performed under pressures of 10 to 20 psi (70-140 kPa) and at temperatures ranging from 82-104° C. In some embodiments, the extraction is performed under pressure at a temperature ranging from about 82° C. to about 88° C., about 82° C. to about 93° C., about 82° C. to about 99° C. about 82° C. to about 104° C.

A temperature of 60° C. is preferred for fruit flavonoids, preparing ayahuasca, cacti, functional mushrooms, leaves, seeds, and bark. For psilocybin-containing fungi the preferred temperature is less than 60° C. For fungi containing muscimol, the temperature is less than 82° C. For Kava, the preferred temperature is 27° C.

### Option B: Pressure

In addition to raising the temperature, elevated pressure can also be used during the extraction step. In one embodiment, the extraction is performed at a pressure ranging from 10 to 20 psi (70-140 kPa). In some embodiments, the extraction is performed at a pressure ranging from about 10 psi (70 kPa) to about 20 psi (140 kPa). In some embodiments, the extraction is performed at a pressure ranging from about 10 psi (70 kPa) to about 12 psi (80 kPa), about 10 psi (70 kPa) to about 14 psi (100 kPa), about 10 psi (70 kPa) to about 16 psi (110 kPa), about 10 psi (70 kPa) to about 18 psi (120 kPa), about 10 psi (70 kPa) to about 20 psi (140 kPa), about 12 psi (80 kPa) to about 14 psi (100 kPa), about 12 psi (80 kPa) to about 16 psi(110 kPa), about 12 psi (80 kPa) to about 18 psi (120 kPa), about 12 psi (80 kPa) to about 20 psi (140 kPa), about 14 psi (100 kPa) to about 16 psi (110 kPa), about 14 psi (100 kPa) to about 18 psi (120 kPa), about 14 psi (100 kPa) to about 20 psi (140 kPa), about 16 psi (110 kPa) to about 18 psi (120 kPa), about 16 psi (110 kPa) to about 20 psi (140 kPa), or about 18 psi (120 kPa) to about 20

psi (140 kPa). In some embodiments, the extraction is performed at a pressure ranging from about 10 psi (70 kPa), about 12 psi (80 kPa), about 14 psi (100 kPa), about 16 psi (110 kPa), about 18 psi (120 kPa), or about 20 psi (140 kPa). In some embodiments, the extraction is performed at a pressure ranging from at least about 10 psi (70 kPa), about 12 psi (80 kPa), about 14 psi (100 kPa), about 16 psi (110 kPa), or about 18 psi (120 kPa). In some embodiments, the extraction is performed at a pressure ranging from at most about 12 psi (80 kPa), about 14 psi (100 kPa), about 16 psi (110 Pa), about 18 psi (120 kPa), or about 20 psi (140 kPa).

### Hydrophilic Compounds

The filtrate or the dried extract comprises, but is not limited to, the following hydrophilic compounds including: cocaine, harman, harmine, harmaline, beta-carbolines, 5-methoxytetrahydroharman, (−)-n(6)-methoxytetrahydroharman, dimethyltryptamine-N(6)-oxide, tetrahydroharmine, telepathine, dihydroshihunine, tetrahydroharman, 6-methoxy-harman, 4-hydroxy-3-5-dimethoxyphenethylamine, dmpea, 4-hydroxy-3-5-dimethoxyphenethylamine, macromerine, coryphanthine, 0-methyl-candicine, corypalmine, n-methyl-corypalmine, 3-methoxytyramine, tyramine, lycaeum, hodgkinsine, psychotridine, 5-ho-dmt, 5-meo-mmt, 5-meo-tryptamine, 1-dopa, serotonin, 5-htp, n-hydroxy-N-methyltryptamine, 2-hydroxy-n-methyltryptamine, Ergot, 4-ho-thc, 4-ho-thcv, 4-ho-cbd, 4-ho-cbg, 4-ho-5-meo-dmt, 4-ho-det, psilocybin, psilocin, baeocystin, norbaeocystin, norpsilocin, aeruginascin, anandamide, hericenones, erinacines, polysaccharides, triterpenoids, peptidoglycans, ganodermic acid, valine, lysine, pa3de, mycosterols, enokipodins, fip-fve, cerevistoral, flammulin, tetraol, ergosterol, ergosta-3-0-glucopyranoside, niacin, cordycepin, adenosine, guanosine, cordymin, polysaccharide peptide, polysaccharide-k, hemagglutinin, muscimol, dmt, 5-meo-dmt, Isa, mescaline, bufotenin, bufotenin oxide, nmt, or any selection or combination selected therefrom. The preferred compounds are psilocybin, psilocin, muscimol, DMT (N,N-Dimethyltryptamine), MAOI (monoamine oxidase inhibitors), harmaline, harmine, mescaline, LSA (d-lysergic acid amide a/k/a Ergine), kavapyrones (kavalactones), mitragynine, 7-hydroxy mitragynine, and cocaine.

### Standardization

The aim is to produce consistent extracts from batch to batch. The extracts are standardized by blending with varying amounts of full-spectrum hydrophilic compounds with different potency levels. Blending can occur before extraction or after extraction. Liquid blends can be made using filtrate, or dry mixes can be made using dried extract.

To standardize the extract, the concentration of hydrophilic compounds and the potency of each component is determined. The component with a high concentration of full spectrum hydrophilic compounds and a high level of potency is blended with another component having a lower amount of hydrophilic compounds and a lower level of potency. Optionally, an inert material may be used for blending. A standardized extract is made by blending the components in the proper ratio to obtain the standard amount.

Standardizing before extraction is done by blending psychoactive biomasses with differing levels of full-spectrum hydrophilic compounds and potencies. The psychoactive biomasses are mixed in the proper ratio for the standardized amount. The blend is then fed into the process to produce an extract with the standard amount.

Standardizing after extraction is done by blending extracts with differing concentrations of full-spectrum hydrophilic compounds and levels of potency. The concentration of full-spectrum hydrophilic compounds and the potency of each extract are determined. Then the extracts are blended in the proper ratio to achieve the standard amount. The dry extract or liquid filtrate may be mixed. Optionally, the extract may be concentrated to increase the concentration of full-spectrum hydrophilic compounds and then blended with another component.

The two psychoactive biomasses can be blended to provide the standard amount and then fed into the process. For example, Psilocybe azurescens, a high potency full spectrum hydrophilic psychoactive biomass, is blended with Psilocybe cubensis, a low potency full spectrum hydrophilic psychoactive biomass. Alternatively, each extract is collected and combined to obtain the standard amount. The liquid filtrates could be blended, the dried extracts could be mixed, or the liquid extracts could be blended and then dried.

The lower concentration component does not have to come from a separate psychoactive biomass. It can come from the same psychoactive biomass. Successive extractions of the same psychoactive biomass will produce extracts with lower concentrations of full-spectrum hydrophilic compounds with lower potency levels. Blending the high-concentration extracts with the lower-concentration extracts provides a standardized amount.

### EXAMPLES

### Example 1: Full Spectrum Coca Leaf Extract

Using the Erythroxylum coca plant leaves, make a slurry using an waterbased solvent of water or water and lemon juice. The slurry is a hot water extraction (about 60° C.) that contains a full spectrum of only water-soluble hydrophilic compounds. Filter the slurry using a 20-micron coffee filter to remove all undissolved plant psychoactive biomass and save the psychoactive biomass for a second extraction. Following filtering of the psychoactive biomass, freeze-dry the filtrate. The freeze-drying process will completely evaporate the water from the hydrophilic compounds leaving behind a powder residue that is pure hydrophilic compounds in a full spectrum composition identical to that of the hydrophilic compound source. The first extract is a strong full-spectrum extract.

Repeat the same steps of extracting and drying a full spectrum of hydrophilic compounds using the same psychoactive biomass used in the first extraction. The second extract is a weak full-spectrum hydrophilic compound extract. Standardize the weak full spectrum extract by adding in a desired amount of the strong full spectrum extract until a desired amount of active hydrophilic compound is achieved. The main active hydrophilic compound is cocaine.

### Example 2: Full Spectrum Psychoactive Fungi Extract

Using the whole body of the Psilocybe azurescens mushroom, prepare a slurry using an waterbased solvent such as water or water and lemon juice. The slurry is an organic hot water (less than 60° C.) extraction that contains a full spectrum of only water-soluble hydrophilic compounds. Filter the slurry using a 20-micron coffee filter to remove all of the undissolved mushroom psychoactive biomass and save the psychoactive biomass for a second extraction. Following filtering of the psychoactive biomass, freeze-dry the filtrate. The freeze-drying process will completely evaporate the water from the hydrophilic compounds leaving behind a powder residue that is pure hydrophilic compounds in a full spectrum composition that is identical to that of the hydrophilic compound source. The first extract is a strong full-spectrum extract. Repeat the same steps of extracting and drying a full spectrum of hydrophilic compounds using the same psychoactive biomass used in the first extraction. The second extract is a weak full-spectrum hydrophilic compound extract. Standardize the weak full spectrum extract by adding in a desired amount of the strong full spectrum extract until a desired amount of active hydrophilic compound is achieved. The main active hydrophilic compounds are psilocin and psilocybin (see Table I).

TABLE I
Extraction of Psilocin and Psilocybin from Psychoactive Fungi

| HPLC Controls | Measured ng/μl | Expected ng/μl | Recovery, % |
|---|---|---|---|
| Psilocin | 117.28 | 125.00 | 93.74 |
| Psilocybin | 127.30 | 125.00 | 101.84 |

### Example 3: Full Spectrum Ayahuasca Extract

Using the vines of the Banisteriopsis caapi and the leaves of Psychotria viridis plants, prepare a slurry using an waterbased solvent such as water or water and lemon juice. The slurry is an organic hot water (about 60° C.) extraction that contains a full spectrum of only water-soluble hydrophilic compounds. Filter the slurry using a 20-micron coffee filter to remove all undissolved plant psychoactive biomass and save the psychoactive biomass for a second extraction. Following filtering of the psychoactive biomass, freeze-dry the filtrate. The freeze-drying process will completely evaporate the water from the hydrophilic compounds leaving behind a powder extract that is pure hydrophilic compounds in a full spectrum composition that is identical to that of the hydrophilic compound source. The first extract is a strong full-spectrum extract. Repeat the same steps of extracting and drying a full spectrum of hydrophilic compounds using the same psychoactive biomass used in the first extraction. The second extract is a weak full-spectrum hydrophilic compound extract. Standardize the weak full spectrum extract by adding in a desired amount of the strong full spectrum extract until a desired amount of active hydrophilic compound is achieved.

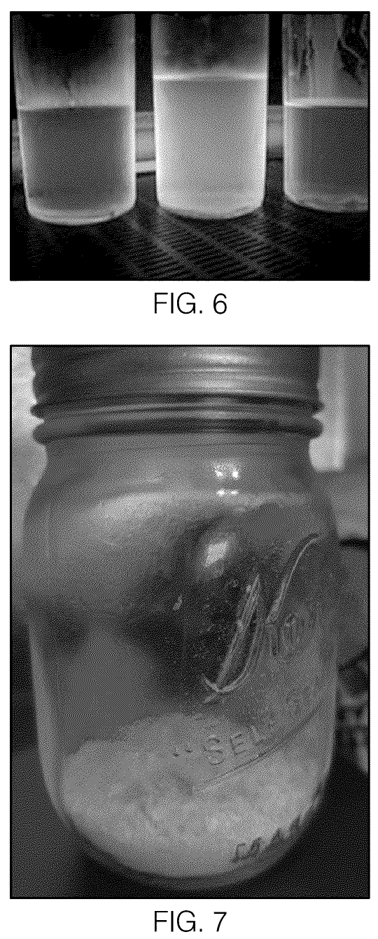

The main active hydrophilic compounds are DMT and beta-carbolines. The presence of DMT is evidenced by the fluorescing of the extract under UV black light. FIG. 6 exhibits the filtrate from three extractions. The middle sample is from the first extraction; its high fluorescence intensity indicates the high concentration of DMT, harmaline, and harmine. The low fluorescence intensity of the repeat extracts on the left shows weak full-spectrum extracts. Lastly, FIG. 7 is an example of the dry extract that fluoresces under UV blacklight.

### Example 4: Full Spectrum San Pedro Cactus Extract

Using the whole column of a San Pedro cactus, make a slurry using an waterbased solvent such as water or water and lemon juice. The slurry is an organic hot water (about 60° C.) extraction that contains a full spectrum of only water-soluble hydrophilic compounds. Filter the slurry using a 20-micron coffee filter to remove all of the undissolved cactus psychoactive biomass and save the psychoactive biomass for a second extraction. Following filtering of the psychoactive biomass, freeze-dry the filtrate. The freeze-drying process will completely evaporate the water from the hydrophilic compounds leaving behind a powder extract that is pure hydrophilic compounds in a full spectrum composition that is identical to that of the hydrophilic compound source. The first extract is a strong full-spectrum extract. Repeat the same steps of extracting and drying a full spectrum of hydrophilic compounds using the same psychoactive biomass used in the first extraction. The second extract is a weak full-spectrum hydrophilic compound extract. Standardize the weak full spectrum extract by adding in a desired amount of the strong full spectrum extract until a desired amount of active hydrophilic compound is achieved. The main active hydrophilic compounds are mescaline and phenylethylamine.

### Example 5: Full Spectrum Hawaiian Baby Woodrose Seed Extract

Using the whole Hawaiian Baby Woodrose Seed, make a cold slurry using an waterbased solvent such as water, water, and lemon juice. The slurry is an organic cold-water extraction that contains a full spectrum of only water-soluble hydrophilic compounds. Filter the slurry using a 20-micron coffee filter to remove all of the undissolved seed psychoactive biomass and save the psychoactive biomass for a second extraction. Following filtering of the psychoactive biomass, freeze-dry the cold brew filtrate. The freeze-drying process will completely evaporate the water from the hydrophilic compounds leaving behind a powder extract that is pure hydrophilic compounds in a full spectrum composition that is identical to that of the hydrophilic compound source. The first extract is a strong full-spectrum extract. Repeat the same steps of extracting and drying a full spectrum of hydrophilic compounds using the same psychoactive biomass used in the first extraction. The second extract is a weak full-spectrum hydrophilic compound extract. Standardize the weak full spectrum extract by adding in a desired amount of the strong full spectrum extract until a desired amount of active hydrophilic compound is achieved. The main active hydrophilic compound is LSA.

### Example 6: Standardization by Blending Psychoactive Biomasses

A slurry was made using 6 to 7.5 grams of pulverized lyophilized Psilocybe cubensis, 0.5 to 2 grams of pulverized lyophilized Psilocybe azurescens, and 300 ml of water. The slurry was heated to a simmer, no greater than 60° C., and agitated for 15 to 30 minutes. The slurry was then filtered through a 20-micron filter. About 300 ml of the filtrate was poured onto a stainless-steel tray and freeze-dried for 24 hours. The dry extract was collected and analyzed by iHILIC-HPLC.

The dry extract was standardized by increasing the amount of baeocystin. Psilocybe cubensis has only trace amounts of baeocystin. The blend, however, indicates the presence of baeocystin. Thus, the baeocystin was extracted from Psilocybe azurescens. Additionally, it is a full spectrum abstract because of the presence of norpsilocin and norbaeocystin (see Table II).

TABLE II
Standardization by Blending Psychoactive Biomass

| HPLC Controls | Measured (ng/μl) | Expected (ng/μl) | Recovery, % |
|---|---|---|---|
| Psilocin | 134.11 | 125 | 107.29 |
| Psilocybin | 139.31 | 125 | 111.45 |
| Norpsilocin | 88.165 | 83.33 | 105.80 |
| Baeocystin | 95.369 | 83.33 | 114.45 |
| Norbaeocystin | 86.931 | 83.33 | 104.32 |

### Example 7: Extraction of Muscimol from Fungi by Conversion of Ibotenic Acid

Muscimol (MC) was extracted from Amanita muscaria by conversion of ibotenic acid (IBA) using acidified water. A quantity of water was acidified with citric acid from lemon juice. The pH of the water was adjusted to a pH of 2.6 using the lemon juice. A slurry was made by contacting a quantity of Amanita muscaria with the acidified water. The slurry was heated to ˜83° C. and held at a rolling simmer for ˜3 hours. The slurry was filtered and freeze-dried, and extract analyzed by HPLC. The results indicated complete conversion of the ibotenic acid (see Table III).

TABLE III
Extraction of Muscimol from Amanita Muscaria
by Conversion of Ibotenic Acid

| | Muscimol, | Ibotenic Acid, | MC + IBA, | Conversion, |
|---|---|---|---|---|
| Sample | mg/g | mg/g | mg/g | mg/g |
| Extract | 41.206 | BLD* | 41.206 | 41.206 |

*BLD = Below the limit of detection.

### DEFINITIONS

“Full Spectrum Extract” is an extract containing all of the primary and secondary metabolites necessary to promote an entourage effect.

“Entourage Effect”—the entourage effect is the natural effect that occurs when all of the primary and secondary metabolites in an organic extract or an organism work together in synergy to produce a desired result unique to each formulation of natural metabolites.

“Hydrophilic Compound” is a chemical compound that is attracted to polar solvents and can be extracted from an organism using a polar solvent.

The disclosure above encompasses multiple distinct inventions with independent utility. While each of these inventions has been disclosed in a particular form, the specific embodiments disclosed and illustrated above are not to be considered in a limiting sense as numerous variations are possible. The subject matter of the inventions includes all novel and non-obvious combinations and sub-combinations of the various elements, features, functions, and/or properties disclosed above and inherent to those skilled in the art pertaining to such inventions. Where the disclosure or subsequently filed claims recite “a” element, “a first” element, or any such equivalent term, the disclosure or claims should be understood to incorporate one or more such elements, neither requiring nor excluding two or more such elements.

Applicant(s) reserves the right to submit claims directed to combinations and sub-combinations of the disclosed inventions that are believed to be novel and non-obvious. Inventions embodied in other combinations and sub-combinations of features, functions, elements, and/or properties may be claimed through amendment of those claims or presentation of new claims in the present application or a related application. Such amended or new claims, whether they are directed to the same invention or a different invention and whether they are different, broader, narrower, or equal in scope to the original claims, are to be considered within the subject matter of the inventions described herein.

1. A method for extracting a full spectrum of hydrophilic compounds comprising: providing a quantity of psychoactive biomass, reducing the size of the psychoactive biomass, making a slurry by contacting the psychoactive biomass with an water-based solvent, steeping the slurry for a period of time, filtering the slurry, collecting the filtrate, drying the filtrate, and collecting the dried extract.

2. The method of claim 1 where the biomass is dry or fresh.

3. The method of claim 1, where the psychoactive biomass comprises at least one species of fungi of the genera: Amanita, Conocybe, Ergot, Galerina, Gymnopilus, Inocybe, Panaeolus, Pholiotina, Plutes or Psilocybe.

4. The method of claim 1, where the psychoactive biomass comprises at least one of the following fungi species: Psilocybe azurescens, Psilocybe bohemica, Psilocybe Baeocystis, Psilocybe cubenis, Psilocybe cyanescens, Psilocybe hoogshagenii, Psilocybe semilanceata, Psilocybe stuntzii, Psilocybe tampanensis, Psilocybe liniformans, Psilocybe weilii, Amanita muscaria, Amanita pantherine, or Claviceps purpurea.

5. The method of claim 1 where the psychoactive biomass comprises at least one of the following plant species: Erythroxylum coca, Psychotria viridis, Diplopterys cabrerana, Psychotria carthagenensis, Mimosa tenuiflora, Peganum harmala, Banisteriopsis caapi, Passiflora incarnata, Kaempferia galangal, Tribulus terrestris, Rivea corymbosa, Ipomoea violacea, Argyreia nervosa, Kava root, or Kratom.

6. The method of claim 1, where the psychoactive biomass comprises at least one species of cacti of the genera: Peyote or Echinopsis.

7. The method of claim 1, where the psychoactive biomass comprises at least one of the following cacti species: Lophophora williamsii, Lophophora decipiens, Echinopsis pachanoi, Echinopsis zamnesiana, or Echinopsis lageniformis.

8. The method of claim 1, where the psychoactive biomass comprises at least one of the following: Sarpa salpa, Kyphosus fuscus, Kyphosus cinerascens, or Kypphosus viagiensis.

9. The method of claim 1 where the extract is dried using air, heat, or lyophilization.

10. The method of claim 1 where the size of the dry psychoactive biomass is reduced to a particle size of less than 6 mm or the fresh biomass is sliced into pieces of less than 0.25 cm in width.

11. The method of claim 1 where the water-based solvent is water, distilled water, purified water, citrus juice, or a blend of citrus juice and water, or a blend of citrus juice and distilled water, or citrus juice and purified water.

12. The method of claim 1 where the slurry is steeped for at least 10 minutes.

13. The method of claim 1 further comprising maintaining the slurry at a temperature of 4 to 85° C.

14. The method of claim 1, where the ratio of water-based solvent to psychoactive biomass is 1:1 to 50:1.

15. The method of claim 1 where the extract comprises at least one of the following: psilocybin, psilocin, muscimol, N,N-Dimethyltryptamine, monoamine oxidase inhibitors, harmaline, harmine, mescaline, d-lysergic acid amide, kavapyrones, mitragynine, 7-hydroxymytragynine, or cocaine.

16. The method of claim 1 where the extract is standardized.

17. A method for extracting a full spectrum of hydrophilic compounds comprising: providing a quantity of psychoactive biomass, drying the psychoactive biomass, reducing the size to less than 6 mm, making a slurry by contacting the psychoactive biomass with a water based solvent, steeping the slurry 10 to 1440 minutes, maintaining the temperature of the slurry at a temperature of 4 to 85° C.; pressurizing the slurry to 70 to 140 kPa, filtering the slurry, collecting the filtrate, drying the filtrate, and collecting the extract.

18. A method for extracting muscimol by conversion of ibotenic acid comprising: providing a quantity of psychoactive biomass, acidifying water with citric acid, making a slurry by contacting the psychoactive biomass with the acidified water, heating the slurry, holding the slurry at a rolling simmer hours, filtering the slurry, drying the filtrate, and collecting the extract.

19. The method of claim 18, where the psychoactive biomass is selected from at least one of the species from the genus Amanita.

20. The method of claim 18, where the psychoactive biomass is selected from at least one of the following species of Amanita: muscaria, pantherine, citrina, gemmeata, pantherine, abietum, porphyria, regalis, strobiliformis.
